(19) United States
(12) Patent Application Publication
(10) Pub. No.: US 2025/0144301 A1
(43) Pub. Date: May 8, 2025

(54) A subassembly of a medicament delivery device

(71) Applicant: SHL Medical AG, Zug (CH)

(72) Inventor: Anders Boström, Nacka Strand (SE)

(21) Appl. No.: 18/836,907

(22) PCT Filed: Jan. 25, 2023
(86) PCT No.: PCT/EP2023/051803

§ 371 Date: Aug. 8, 2024

(30) Foreign Application Priority Data
Feb. 8, 2022 (EP) 22155679.8

(51) Int. Cl. A61M 5/315

(52) U.S. Cl. CPC A61M 5/31501

(57) ABSTRACT

The present disclosure provides a subassembly of a medicament delivery device configured to expel medicament from a medicament container, the subassembly comprising a housing having a proximal end and a distal end; a tubular plunger rod operatively arranged to, upon activation, act on the medicament container, the tubular plunger rod assumes a pre-tensioned state by a drive spring; an actuator housing comprising at least one holding member arranged to lock the plunger rod in the pre-tensioned state; and a plunger holder having a blocking surface, the plunger holder being operatively configured to move distally from a blocking position in which the at least one holding member abuts against the blocking surface to prevent the at least one holding member from releasing the plunger rod from its pre-tensioned state, to a non-blocking position in which the blocking surface is distant from the position restricting movement of the holding member; wherein the drive spring is at least partly housed in the tubular plunger rod and is operatively connected to the distal end of the housing.

### CROSS-REFERENCE TO RELATED APPLICATIONS

The present application is a U.S. National Phase Application pursuant to 35 U.S.C. § 371 of International Application No. PCT/EP2023/051803 filed Jan. 25, 2023, which claims priority to European Patent Application No. 22155679.8 filed Feb. 8, 2022. The entire disclosure contents of these applications are herewith incorporated by reference into the present application.

### TECHNICAL FIELD

The present disclosure generally relates to a subassembly of a medicament delivery device configured to expel medicament from a medicament container.

### BACKGROUND

A number of medical conditions require reliable provision of a medicament from a medicament delivery device. The medicament is typically comprised in a medicament container within the medicament delivery device, the medicament container being configured to expel the medicament via some type of delivery member, such as a needle or a nozzle.

These days, a number of different medicament delivery devices exist, including various types of injectors (for example pen injectors, autoinjectors, on-body devices). Although many of these devices have enabled major improvements in the management of a number of medical conditions, various drawbacks do still exist in the current technology.

Many medicament delivery devices comprise components which are movable relative each other. One type of relative movement between components may activate the medicament delivery device, e.g. exposing the delivery member or initiating expel of medicament from the medicament delivery device. Another type of relative movement between components may result in that two components are secured relative each other, e.g. by axially or radially fixating one component to another, or by means of a locking element. The relative fixation of components to avoid relative movement is often desired prior to using the medicament delivery device. That is, in the default state of the medicament delivery device, relative movement between components are undesired, as such relative movement may result in an unintentional action of the medicament delivery device. Such unintentional actions may e.g. be the result of unsatisfactory relative fixation of components. In considering these problems, the applicant has appreciated that various developments could be made to help improve the medicament delivery devices on the market today, which are set out in more detail below.

### SUMMARY

The invention is defined by the appended claims, to which reference should now be made. An object of the present disclosure is thus to provide a medicament delivery device which solves, or at least mitigates, problems of the prior art.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to a first aspect of the present disclosure there is provided a subassembly of a medicament delivery device configured to expel medicament from a medicament container, the subassembly comprising: a housing having a proximal end and a distal end; a tubular plunger rod operatively arranged to, upon activation, act on the medicament container for expelling a medicament, the tubular plunger rod assumes a pre-tensioned state by a drive spring; an actuator housing fixedly attached to the housing, the actuator housing comprising at least one holding member arranged to lock the plunger rod in the pre-tensioned state; and a plunger holder having a blocking surface, the plunger holder being operatively configured to move distally from a blocking position in which the at least one holding member abuts against the blocking surface to prevent the at least one holding member from releasing the plunger rod from its pre-tensioned state, to a non-blocking position in which the blocking surface is distant from the position restricting movement of the holding member; wherein the drive spring is at least partly housed in the tubular plunger rod and is operatively connected to the distal end of the housing.

An advantageous effect which may be obtained herein is that the plunger rod is securely prevented from being activated until the plunger holder has moved distally to the non-blocking position. The distal movement of the plunger rod from the blocking position to the non-blocking position is preferably executed after a specific sequence order of usage, whereby the risk of accidentally activating the tubular plunger rod at a point in time when a medicament recipient of the medicament is not prepared for the medicament injection can be reduced.

Also, the use of a tubular plunger rod in which the drive spring is at least partly housed can enable for a further degree of freedom when designing an interface between the tubular plunger rod and the actuator housing. In detail, no drive spring interferes between the tubular plunger rod and the actuator housing.

According to an example embodiment, the at least one holding member may comprise a proximally extending holding arm arranged to lock the plunger rod in the pre-tensioned state.

According to an example embodiment, the proximally extending holding arm may be radially displaceable in a direction away from the plunger rod when the plunger holder assumes the non-blocking position. Hereby, when the plunger holder assumes the non-blocking position the drive spring exerts an axial force on the tubular plunger holder, thereby forcing the proximally extending holding arm to displace radially outwards in a direction away from an axially extending geometric center axis of the subassembly.

According to an example embodiment, the proximally extending holding arm may comprise a surface portion inclined radially towards the tubular plunger rod in the proximal direction. Thus, and as briefly indicated above, the drive spring exerts an axial force on the tubular plunger holder, whereby an engagement member, which is described further below, of the tubular plunger rod acts on the inclined surface portion for forcing the proximally extending holding arm to move radially.

According to an example embodiment, the at least one holding member may comprise a circumferentially arranged holding ring having a protrusion extending radially towards the plunger rod, the protrusion being arranged to lock the plunger rod in the pre-tensioned state.

According to an example embodiment, the circumferentially arranged holding ring may be radially displaceable in a direction away from the plunger rod when the plunger holder assumes the non-blocking position.

The circumferentially arranged holding ring is thus advantageously enclosing the tubular plunger rod.

According to an example embodiment, the tubular plunger rod may comprise an engagement member configured to engage with the holding member when the plunger holder assumes the blocking position.

According to an example embodiment, the engagement member may comprise a protrusion arranged on an envelope surface of the tubular plunger rod, the protrusion extending in a radial and an axial direction. Hereby, an advantageous interface for locking the plunger rod to the actuator housing is provided.

According to an example embodiment, the protrusion may comprise a surface portion inclined radially and distally. The surface portion of the protrusion which is inclined radially and distally hereby interacts with the above described surface portion of the holding arm. In yet further detail, the surface portions are in abutment with each other when the plunger holder assumes the blocking position and slide relative to each other when the plunger holder has transitioned from the blocking position to the non-blocking position.

According to an example embodiment, the engagement member may comprise an indentation in an envelope surface of the tubular plunger rod, the holding member being configured to engage with the indentation when the plunger holder assumes the blocking position. Hereby, the holding member is securely attached into the indentation.

According to an example embodiment, the indentation may be a through hole.

According to an example embodiment, the subassembly may further comprise a tubular ring member housed by, and fixedly attached to, the actuator housing, the tubular ring member comprising a resilient tongue having an abutment surface arranged in abutment with an envelope surface of the tubular plunger rod. The resilient tongue is preferably arranged to engage with a protrusion of the tubular plunger rod upon activation of the tubular plunger rod, whereby a click sound will be generated that intuitively informing the user that medicament is injected.

According to an example embodiment, the resilient tongue may extend radially and circumferentially towards the tubular plunger rod.

According to an example embodiment, the subassembly may further comprise a delivery member cover arranged in the housing and extending proximally from the proximal end of the housing, the delivery member cover being configured to, during the medicament delivery operation, move linearly relative to the housing from a protracted position to a retracted position in which the delivery member cover is received further in the housing.

The delivery member cover can advantageously cover a needle of the medicament delivery before usage. When pressing the medicament delivery device, and in particular the delivery member cover, onto e.g. the skin of a medicament recipient, the delivery member cover is pushed into housing of the medicament delivery device.

According to an example embodiment, the delivery member cover may comprise a distal end portion having a pushing surface arranged to abut the plunger holder when the delivery member cover is moved to its retracted position to thereby move the plunger holder to its non-blocking position.

Hereby, the delivery member cover can advantageously push the plunger holder to its non-blocking position, whereby the tubular plunger rod can be released at a point in time when the user expects the tubular plunger rod to be released.

According to an example embodiment, the subassembly may further comprise a safety clip releasably attached to the housing, the safety clip comprises a first radially extending hook, wherein the safety clip is operatively arranged to, upon activation, move from a first state in which the first radially extending hook is abutting a distal end of the plunger holder, to a second state in which the first radially extending hook is displaced radially away from the plunger holder to thereby allow the plunger holder to transition from the blocking position to the non-blocking position.

The safety clip can thus advantageously prevent the plunger holder from being accidentally arranged in the non-blocking position.

According to an example embodiment, the safety clip may further comprise a second radially extending hook abutting the pushing surface when the safety clip assumes the first state and radially displaced from the pushing surface when the safety clip assumes the second state.

A potential advantage is that an additional safety mechanism is provided for preventing the plunger holder to be accidentally arranged in the non-blocking position since both the delivery member cover as well as the plunger holder are kept fixed by the first and second radially extending hooks until the safety clip assumes the second state.

According to an example embodiment, the safety clip may be releasably attached to the distal end of the housing and extends proximally along an envelope surface of the housing when the safety clip assumes the first state.

According to an example embodiment, the safety clip may be releasably attached to the distal end of the housing and extends in a circumferential direction along an envelope surface of the housing when the safety clip assumes the first state.

According to a second aspect, there is provided a medicament delivery device for expelling medicament from a medicament container via a delivery member, the medicament delivery device comprising a subassembly according to any one of embodiments described above in relation to the first aspect.

Effects and features of the second aspect are largely analogous to those described above in relation to the first aspect.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 schematically shows a perspective view of an example of a medicament delivery device;

FIG. 2 shows an exploded view of the medicament delivery device in FIG. 1;

FIGS. 3A-3D illustrate the medicament delivery device in cross-section during a medicament delivery operation;

FIG. 4A schematically shows a perspective view of a tubular plunger rod according to an example embodiment;

FIG. 4B schematically shows a perspective view of a holding ring according to an example embodiment;

FIGS. 5A-5B schematically illustrate the interaction between the tubular plunger rod in FIG. 4A and the holding ring in FIG. 4B;

FIG. 6A is a cross-sectional view illustrating the medicament delivery device according to another example embodiment;

FIGS. 6B-6C illustrate a portion of the medicament delivery device in detailed cross-section during a medicament delivery operation according to another example embodiment;

FIG. 7 schematically shows a perspective view of a tubular ring member according to an example embodiment;

FIGS. 8A-8B schematically show a safety clip according to an example embodiment; and

FIG. 9 is a cross-sectional view illustrating the interaction between the safety clip in FIGS. 8A-8B and the plunger holder.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

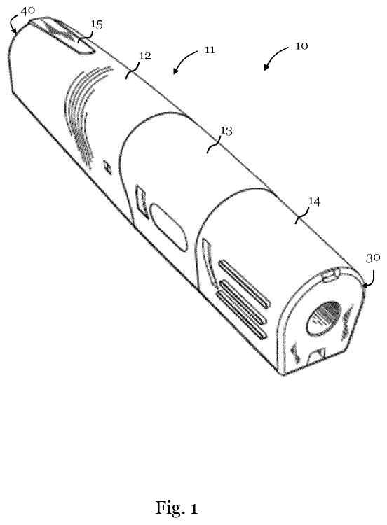

FIG. 1 shows a perspective view of a prior art medicament delivery device 10. The particular medicament delivery device 10 illustrated in FIG. 1 is an injector configured to inject a dose of medicament into the body of a user via a syringe arranged in a housing 11. The housing comprises a back outer shell 12 and front outer shell 13 of the injector 10 after a protective front cap 14 has been removed.

The injector 10 is further arranged with a safety clip 15 which must be removed by the user before use of the injector 10, since a spring-loaded plunger rod arranged in the injector 10 otherwise cannot be released to cause medicament delivery.

An end of the injector 10 facing towards the user is referred to as proximal end 30 while an end of the injector 10 facing away from the user is referred to as distal end 40.

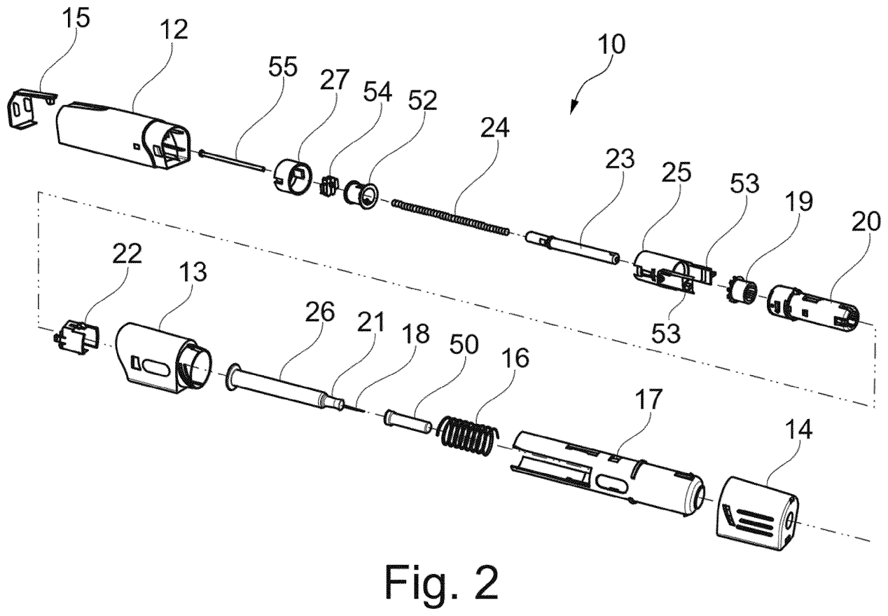

FIG. 2 shows individual components of the injector 10 of FIG. 1 in an exploded view. The back outer shell 12 and the front outer shell 13 house all components except for the safety clip 15 which is attached to an exterior of the back outer shell 12, while the front cap 14 is snapped into attachment with a front end of the front outer shell 13 for protecting the user from a syringe as will be described in more detail in the following.

The injector 10 comprises a delivery member cover spring 16 arranged to spring-load a delivery member cover 17 utilized to protect the user from the syringe 18. The syringe 18 is forming part of a medicament container 26, also in the following referred to as a syringe. The delivery member cover spring 16 may also be referred to as a needle cover spring 16 and the delivery member cover 17 may also be referred to as a needle cover 17. Further associated with the medicament container 26 is a syringe support 19 and a syringe carrier 20 (and medicament 21 to be delivered by the injector 10). A lock out collar 22 is employed to prevent the needle cover 17 from again being pushed towards a distal end position once medicament has been delivered and the needle cover 17 has reverted to a position at the proximal end 30 of the injector 10 for covering the syringe 18.

The injector 10 further comprises a tubular plunger rod 23 which is spring-loaded with a drive spring 24 against a distal end wall of an actuator housing 25 for pushing the medicament 21 through the syringe 18 for delivery to the user upon plunger rod spring release. The drive spring 24 is arranged inside the tubular plunger rod 23. As will be described in further detail below, the actuator housing 25 comprises at least one holding member (not shown in FIG. 2). Further, the actuator housing 25 is fixedly attached to the housing 11 by a pair of actuator housing arms 53.

A plunger holder 27 is axially movable from a blocking position in which the plunger rod 23 is held in a position where the drive spring 24 is compressed, i.e. the tubular plunger rod 23 assumes a pre-tensioned state, to a non-blocking position where the plunger holder 27 is moved by the needle cover 17 towards a distal end, and the drive spring 24 is released, thereby causing spring release to have the plunger rod 23 push the medicament 21 through the syringe 18 for delivery. The above briefly described holding member of the actuator housing 25 is arranged to lock the tubular plunger rod 23 to assume the pre-tensioned state. However, to cause spring release, the user must first remove the safety clip 15 which holds the plunger holder 26 in the lock position when being attached to the back outer shell 12.

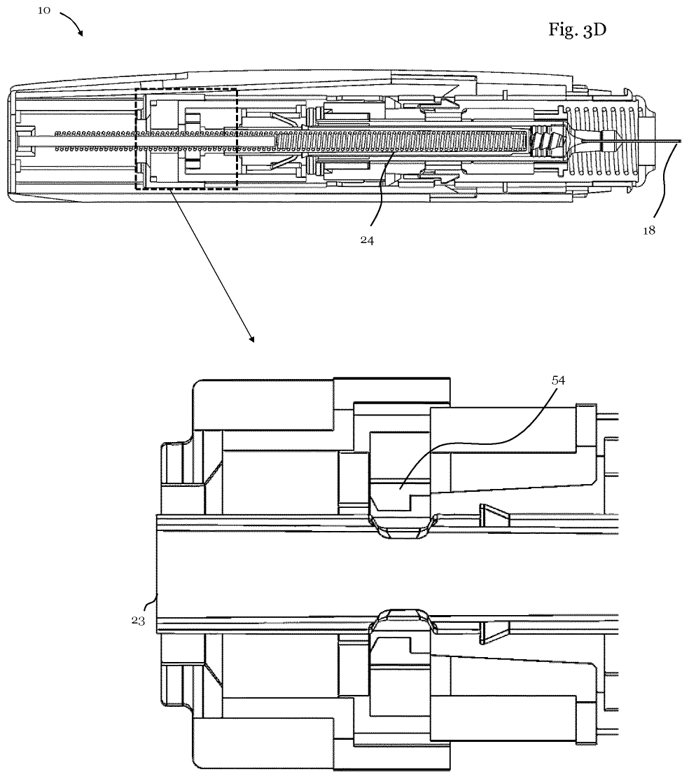

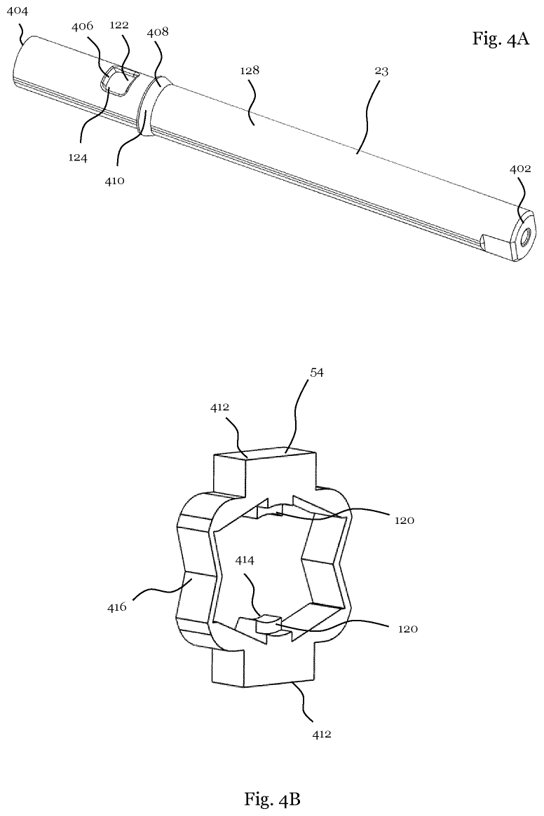

As depicted in FIG. 2, the medicament delivery device 10 further comprises a medicament delivery cover 50 (needle cover) protecting a needle (in this case the needle of a syringe) before removal of the protective front cap 14, and a tubular ring member 52 housed by, and fixedly attached to, the actuator housing 25. Also, the holding member comprises a holding ring 54 which is described in further detail below with reference to the description of FIG. 4B. Moreover, the medicament delivery device 10 comprises a spring guide rod 55 around which the drive spring 24 is arranged. The spring guide rod 55 is attached to the interior rear end of the housing, i.e. at the distal end 40 at an inner surface of the back outer shell 12.

The following description will present further details of the activation mechanisms for causing the tubular plunger rod to move from the pre-tensioned state to release of the tubular plunger rod.

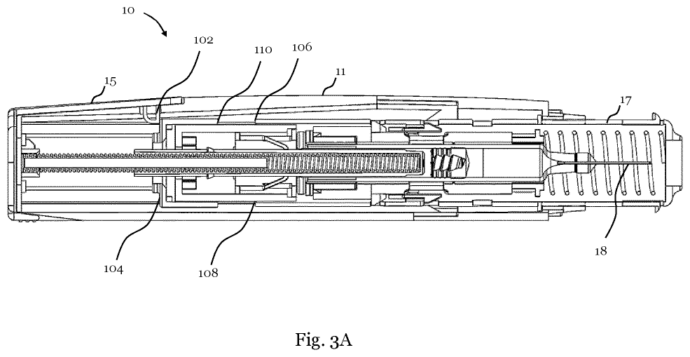

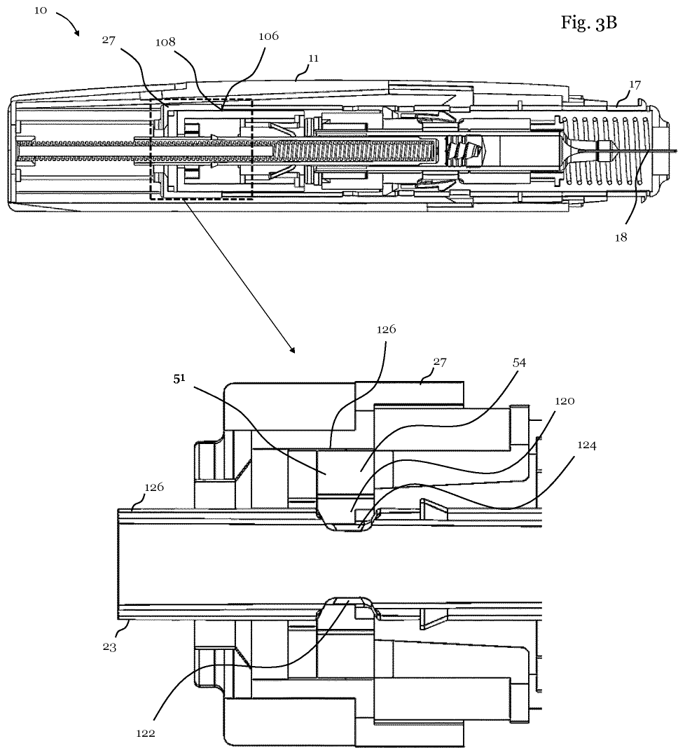

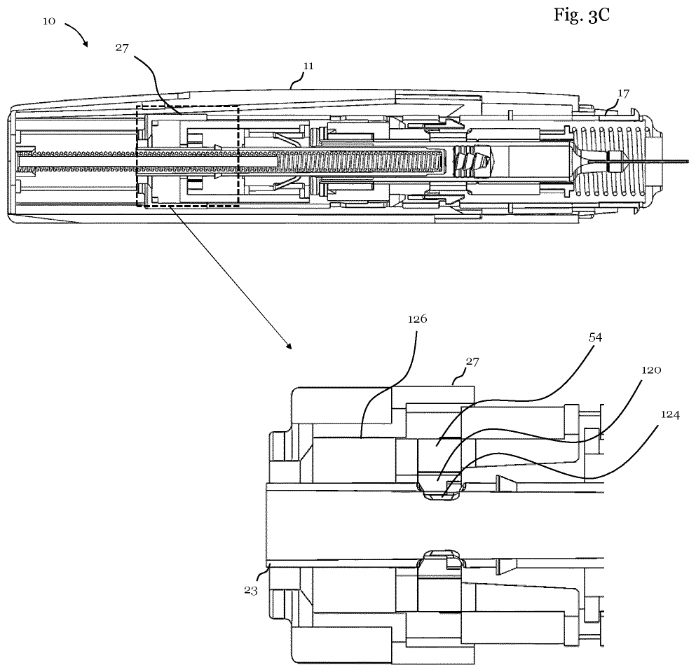

Reference is initially made to FIGS. 3A-3D which illustrate the medicament delivery device 10 in cross-section during a medicament delivery operation. Starting with FIG. 3A, the protective front cap (14 in FIGS. 1 and 2) has been removed and the syringe 18 is fully housed inside the delivery member cover 17. Thus, in the configuration of FIG. 3A, a user of the medicament delivery device 10 is protected from being accidentally stung by a needle of the syringe 18. As is also depicted in FIG. 3A, the safety clip 15 assumes a first state in which a first radially extending hook 102 of the safety clip 15 is abutting a distal end 104 of the plunger holder 27. Hereby, the plunger holder 27 is prevented from moving distally. The delivery member cover 17 comprises a distal end portion 106 provided with a pushing surface 108 which in the FIG. 3A configuration is axially spaced apart from a proximal end 110 of the plunger holder 27.

Turning to FIG. 3B, the safety clip 15 illustrated in FIG. 3A has been removed from the housing 11, thereby allowing the plunger holder 27 to move distally relative to the housing 11.

Furthermore, the delivery member cover 17 has moved linearly in the distal direction relative to the housing 11 from the protracted position illustrated in FIG. 3A towards a retracted position which is illustrated in FIG. 3C. In detail, the delivery member cover 17 has moved distally to a position in which the pushing surface 108 is abutting the proximal end 110 of the plunger holder 27. However, the delivery member cover 17 is in FIG. 3B still not pushing the plunger holder 27 distally.

As can be seen in the enlarged portion of FIG. 3B, the above described holding member 51 comprises the holding ring 54. The holding ring 54 comprises a protrusion 120 extending radially towards the plunger rod 23. In further detail, the protrusion 120 engages with an engagement member 122 of the tubular plunger rod 23. As can be seen, and described in further detail below with reference to FIG. 4, the engagement member 122 comprises an indentation 124 in an envelope surface 128 of the tubular plunger rod 23. The indentation 124 is preferably arranged as a through hole in the envelope surface 128 of the plunger rod 23.

Furthermore, the plunger holder 27 comprises a blocking surface 126. In FIG. 3B, the plunger holder 27 assumes a blocking position in which the blocking surface 126 and the holding ring 54 are in abutment with each other. Hereby, the blocking surface 126 prevents the holding ring 54 to move radially outward. The protrusion 120 of the holding ring 54 thus maintains the plunger rod 23 in the pre-tensioned state.

Turning to FIG. 3C in which the delivery member cover 17 has moved further distally compared to the configuration depicted in FIG. 3B. The delivery member cover 17 has in FIG. 3C moved the plunger holder 27 distally relative to the housing 11. As is detailed in the enlarged portion of FIG. 3C, the plunger holder 27 has been moved distally such as to assume a non-blocking position. In the non-blocking position, the blocking surface 126 is moved distally from the position restricting the radial movement of the holding ring 54. In further detail, the blocking surface 126 is not interfering with the radial displacement of the holding ring 54. The protrusion 120 of the holding ring 54 can now be released from the indentation 124 of the plunger rod 23.

Reference is now made to FIG. 3C which schematically illustrates an injection finish of medicament through the syringe 18. When the plunger holder 27 assumes the non-blocking position as described above in relation to FIG. 3C, the force acting on the plunger rod 23 by the drive spring 24 forces the holding ring 54 to move in a radially outward direction, thereby releasing the plunger rod 23 in the proximal direction. The plunger rod 23 hereby pushes the medicament 21 through the syringe 18 for delivery to the user.

In order to describe the tubular plunger rod 23 and the holding ring 54 in further detail, reference is now made to FIGS. 4A and 4B. Starting with FIG. 4A which illustrates the tubular plunger rod 23 according to an example embodiment. As described above, the plunger rod 23 is tubular, i.e. it is formed as a tube or cylinder having a closed proximal end 402 and an open distal end 404. The proximal end 402 is thus configured to push the medicament 21 through the syringe 18 when releasing the plunger rod 23 as described above.

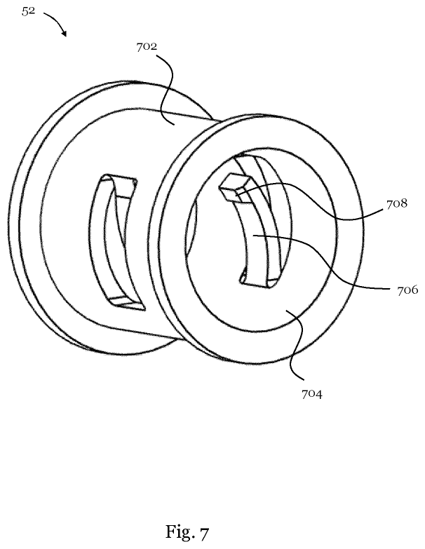

The plunger rod 23 comprises the above described engagement member 122, which is preferably arranged as an indentation 124 in the form of a through hole in the envelope surface 128 of the plunger rod 23. As is better illustrated in e.g. FIGS. 3B-3D, the plunger rod 23 comprises a pair of indentations 124 on opposite radial sides. A distal end of the indentation 124 comprises a surface portion 406. The surface portion 406 is inclined radially and distally. In further detail, the surface portion 406 extends distally from a radially inner end towards a radially outer end. The tubular plunger rod 23 also comprises a circumferentially extending protrusion 408. The circumferentially extending protrusion 408 is arranged proximally from the engagement member 122 and comprises a protrusion surface 410 extending from the envelope surface 128 radially and distally. The circumferentially extending protrusion 408 is configured to interact with a resilient tongue of a tubular ring member as will be described in further detail below with reference to FIG. 7.

Turning to FIG. 4B which illustrates the holding ring 54 according to an example embodiment. The holding member 51 comprises the holding ring 54. As can be seen in FIG. 4B, the holding ring is a circumferentially arranged holding ring 54 which surrounds, i.e. enclose, the tubular plunger rod 23. The holding ring 54 comprises a holding ring surface 412 arranged in abutment with the blocking surface 126 of the plunger holder 27 when plunger holder 27 assumes the blocking position.

The holding ring 54 further comprises the above described protrusion 120. The protrusion 120 extends radially towards the plunger rod 23 and is arranged to lock the plunger rod 23 in the pre-tensioned state when the plunger holder 27 assumes the blocking position. The protrusion 120 comprises a holder surface 414 arranged in abutment with the surface portion 406 of the plunger rod indentation 124. The holder surface 414 is inclined radially and proximally. In further detail, the holder surface 414 extends proximally in a radially extension towards an axial center of the holding ring 54.

Furthermore, the holding ring 54 also comprises a resilient portion 416. The resilient portion 416 enables the holding ring to disengage from the indentation 124 when the plunger holder 27 transitions form the blocking position to the non-blocking position.

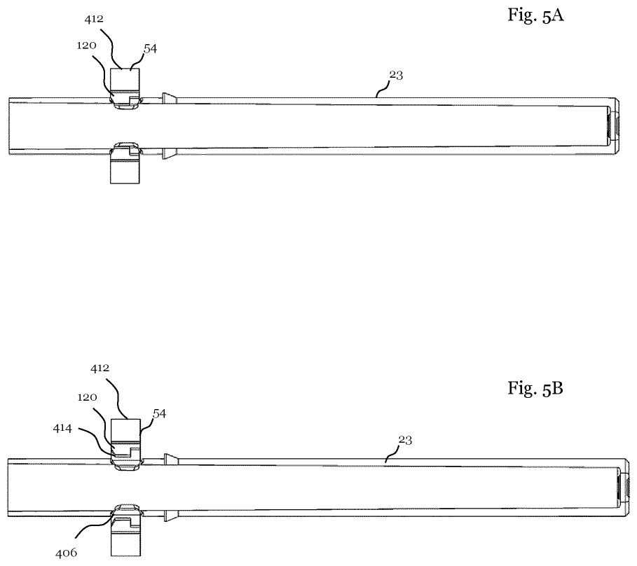

Turning now to FIG. 5A-5B, in which FIG. 5A illustrates the plunger rod 23 and the holding ring 54 when the plunger holder assumes the blocking position, while FIG. 5B illustrates the plunger rod 23 and the holding ring 54 when the plunger holder assumes the non-blocking position. As can be seen in FIG. 5A, the protrusion 120 of the holding ring 54 engages with the indentation 124 of the plunger rod 23. When the plunger holder 27 transitions from the blocking position to the non-blocking position, the protrusion 120, as well as the holding ring surface 412 is displaced radially. In detail, the axial force exerted by the drive spring 24 on the plunger rod 23 generates a radial force component between the surface portion 406 of the plunger rod 23 and the holder surface 414 of the holding ring 54 forcing the resilient portion 416 to move the protrusion 120 radially away from the plunger rod 23.

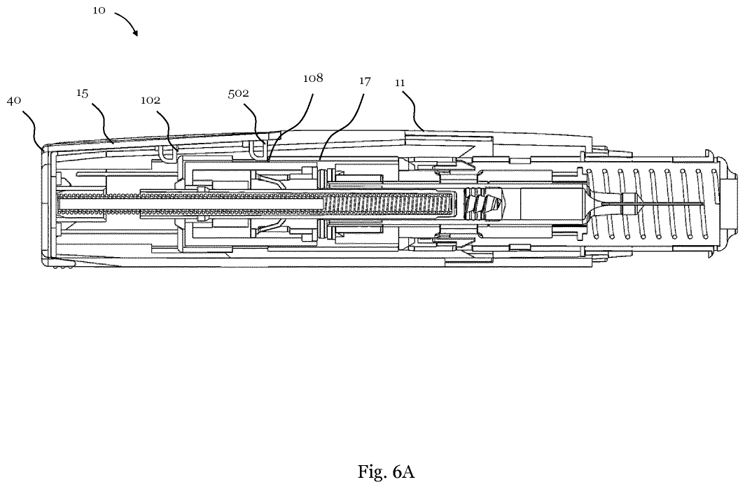

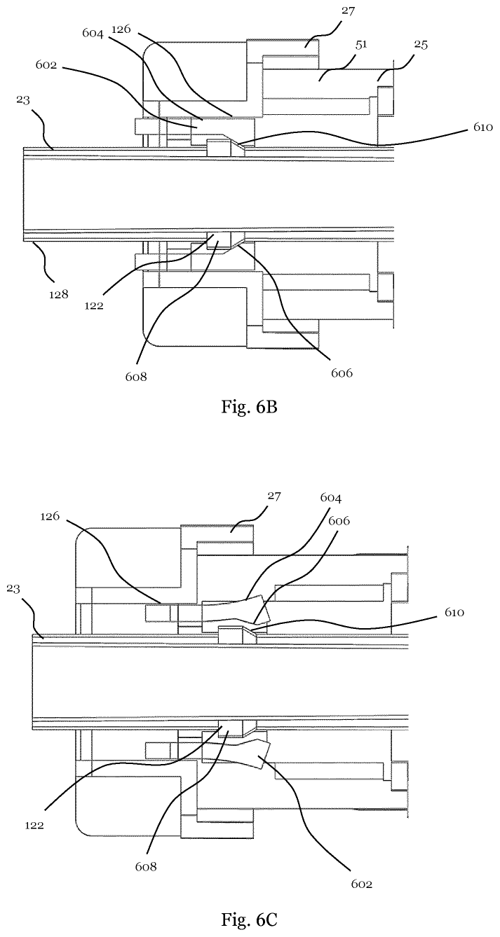

In order to describe the medicament delivery device 10 according to a further example embodiment, reference is now made to FIGS. 6A-6C. As can be seen in FIG. 6A, the safety clip 15 comprises the above described first radially extending hook 102. The safety clip 15 also comprises a second radially extending hook 502. The safety clip 15 extends proximally from the distal end 40 of the medicament delivery device and the first radially extending hook 102 interacts with the distal end 104 of the plunger holder 27 in a similar manner as described above. The second radially extending hook 502 on the other hand is arranged in abutment with the pushing surface 108 of the delivery member cover 17. Accordingly, before the safety clip is removed from the housing 11, the delivery member cover 17 is unable to move distally when exerted by a force on its proximal end.

Turning to FIGS. 6B-6C, the holding member 51 of the actuator housing 25 comprises a proximally extending holding arm 602. The holding arm 602 comprises is arranged to lock the plunger rod in the pre-tensioned state when the plunger holder 27 assumes the blocking position. In further detail, the holding arm 602 comprises a holding arm surface 604 in abutment with the blocking surface 126, whereby the holding arm 602 is prevented from being radially displaced. The holding arm 602 also comprises a surface portion 606 inclined radially towards the tubular plunger rod in the proximal direction. When the plunger holder 27 assumes the blocking position as illustrated in FIG. 6B, the surface portion 606 is abutting a protrusion 608 of the engagement member 122 of the plunger rod 23. The protrusion 608 is arranged on the envelope surface 128 of the plunger rod 23 and extends radially and axially. Further, the protrusion 608 comprises a surface portion 610 inclined radially and distally. Hence, the surface portion 606 of the holding arm 602 is abutting the surface portion 610 of the protrusion 608 when the plunger holder 27 assumes the blocking position.

In FIG. 6C, the plunger holder assumes the non-blocking position. The blocking surface 126 is thus moved distally in a similar manner as described above for the embodiment illustrated in FIGS. 3A-3D. By means of the configuration of the surface portion 606 of the holding arm 602 and the surface portion 610 of the protrusion 608, and by the fact that the holding arm surface 604 is not interacting with the blocking surface 126, the holding arm 602 is forced radially away from the engagement member 122 by the axial force exerted on the plunger rod 23 by the drive spring 24.

Reference is now made to FIG. 7 for describing the above briefly mentioned tubular ring member 52. The tubular ring member 52 is housed by, and fixedly attached to, the actuator housing 25. The tubular ring member comprises an outer envelope surface 702 facing the actuator housing 25 and an inner envelope surface 704 facing the plunger rod 23. The inner envelope surface 704 comprises a resilient tongue 706. The resilient tongue 706 comprises an abutment surface 708 which is in abutment with the envelope surface 124 of the plunger rod 23. As illustrated in FIG. 7, the resilient tongue 706 extends radially and circumferentially from the inner envelope surface 704 towards the plunger rod 23.

When the plunger rod is released as depicted in e.g. FIGS. 3C-3D, the abutment surface 708 will slide over the protrusion surface 410 depicted in FIG. 4A, or over the protrusion 608 depicted in FIGS. 6B-6C, thereby generating an intuitive click sound for the user of the medicament delivery device indicating that medicament is being injected.

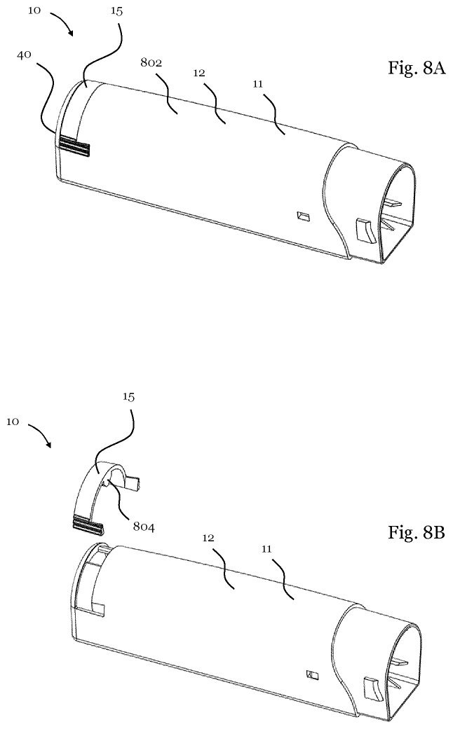

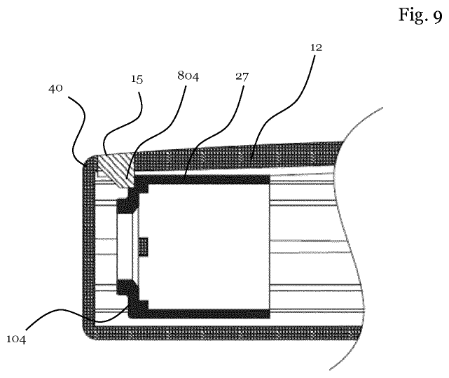

Reference is now made to FIGS. 8A-8B and FIG. 9 for describing the safety clip 15 according to another example embodiment. In a similar vein as the safety clip 15 depicted in e.g. FIG. 1, the safety clip 15 in FIGS. 8A-8B and FIG. 9 is releasably attached to the distal end 40 of the housing. However, the safety clip 15 in FIGS. 8A-8B and FIG. 9 extends in a circumferential direction along an envelope surface 802 of the housing 11 when the safety clip assumes the first state. In yet further detail, the safety clip 15 is arranged on the back outer shell 12 of the housing 11.

The safety clip 15 comprises a radially protruding portion 804 protruding radially in a direction away from the envelope surface 802 of the housing 11. As can be seen in FIG. 9, the radially protruding portion 804 is in abutment with the distal end 104 of the plunger holder 27 when the safety clip assumes the first state. Hereby, the plunger holder 27 is prevented from being displaced distally until the safety clip 15 is removed. As can be seen in FIG. 8B, the safety clip 15 is removed by radially displacing the safety clip 15 from the housing 11.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are outlined in the following clauses.
1. A subassembly of a medicament delivery device configured to expel medicament from a medicament container, the subassembly comprising:
   a housing having a proximal end and a distal end;
   a tubular plunger rod operatively arranged to, upon activation, act on the medicament container for expelling a medicament, the tubular plunger rod assumes a pre-tensioned state by a drive spring;
   an actuator housing fixedly attached to the housing, the actuator housing comprising at least one holding member arranged to lock the plunger rod in the pre-tensioned state; and
   a plunger holder having a blocking surface, the plunger holder being operatively configured to move distally from a blocking position in which the at least one holding member abuts against the blocking surface to prevent the at least one holding member from releasing the plunger rod from its pre-tensioned state, to a non-blocking position in which the blocking surface is distant from the position restricting movement of the holding member;
   wherein the drive spring is at least partly housed in the tubular plunger rod and is operatively connected to the distal end of the housing.
2. The subassembly according to clause 1, wherein the at least one holding member comprises a proximally extending holding arm arranged to lock the plunger rod in the pre-tensioned state.
3. The subassembly according to clause 2, wherein the proximally extending holding arm is radially displaceable in a direction away from the plunger rod when the plunger holder assumes the non-blocking position.
4. The subassembly according to any one of clauses 2 or 3, wherein the proximally extending holding arm comprises a surface portion inclined radially towards the tubular plunger rod in the proximal direction.
5. The subassembly according to clause 1, wherein the at least one holding member comprises a circumferentially arranged holding ring having a protrusion extending radially towards the plunger rod, the protrusion being arranged to lock the plunger rod in the pre-tensioned state.
6. The subassembly according to clause 5, wherein the circumferentially arranged holding ring is radially displaceable in a direction away from the plunger rod when the plunger holder assumes the non-blocking position.
7. The subassembly according to any one of the preceding clauses, wherein the tubular plunger rod comprises an engagement member configured to engage with the holding member when the plunger holder assumes the blocking position.
8. The subassembly according to clause 7, wherein the engagement member comprises a protrusion arranged on an envelope surface of the tubular plunger rod, the protrusion extending in a radial and an axial direction.
9. The subassembly according to clause 8, wherein the protrusion comprises a surface portion inclined radially and distally.
10. The subassembly according to clause 7, wherein the engagement member comprises an indentation in an envelope surface of the tubular plunger rod, the holding member being configured to engage with the indentation when the plunger holder assumes the blocking position.
11. The subassembly according to clause 10, wherein the indentation is a through hole.
12. The subassembly according to any one of the preceding clauses, wherein the subassembly further comprises a tubular ring member housed by, and fixedly attached to, the actuator housing, the tubular ring member comprising a resilient tongue having an abutment surface arranged in abutment with an envelope surface of the tubular plunger rod.
13. The subassembly according to clause 12, wherein the resilient tongue extends radially and circumferentially towards the tubular plunger rod.
14. The subassembly according to any one of the preceding clauses, wherein the subassembly further comprises a delivery member cover arranged in the housing and extending proximally from the proximal end of the housing, the delivery member cover being configured to, during the medicament delivery operation, move linearly relative to the housing from a protracted position to a retracted position in which the delivery member cover is received further in the housing.
15. The subassembly according to clause 14, wherein the delivery member cover comprises a distal end portion having a pushing surface arranged to abut the plunger holder when the delivery member cover is moved to its retracted position to thereby move the plunger holder to its non-blocking position.
16. The subassembly according to any one of the preceding clauses, wherein the subassembly further comprises a safety clip releasably attached to the housing, the safety clip comprises a first radially extending hook, wherein the safety clip is operatively arranged to, upon activation, move from a first state in which the first radially extending hook is abutting a distal end of the plunger holder, to a second state in which the first radially extending hook is displaced radially away from the plunger holder to thereby allow the plunger holder to transition from the blocking position to the non-blocking position.
17. The subassembly according to clause 16 when dependent on clause 15, wherein the safety clip further comprises a second radially extending hook abutting the pushing surface when the safety clip assumes the first state and radially displaced from the pushing surface when the safety clip assumes the second state.
18. The subassembly according to any one of clauses 16-17, wherein the safety clip is releasably attached to the distal end of the housing and extends proximally along an envelope surface of the housing when the safety clip assumes the first state.
19. The subassembly according to any one of clauses 16-17, wherein the safety clip is releasably attached to the distal end of the housing and extends in a circumferential direction along an envelope surface of the housing when the safety clip assumes the first state.
20. A medicament delivery device for expelling medicament from a medicament container via a delivery member, the medicament delivery device comprising a subassembly according to any one of the preceding clauses.

1-15. (canceled)

16. A subassembly of a medicament delivery device configured to expel medicament from a medicament container, the subassembly comprising:
a housing having a proximal end and a distal end;
a tubular plunger rod operatively arranged to, upon activation, act on the medicament container for expelling a medicament, the tubular plunger rod assumes a pre-tensioned state by a drive spring;
an actuator housing fixedly attached to the housing, the actuator housing comprising at least one holding member arranged to lock the plunger rod in the pre-tensioned state; and
a plunger holder having a blocking surface, the plunger holder being operatively configured to move distally from a blocking position in which the at least one holding member abuts against the blocking surface to prevent the at least one holding member from releasing the plunger rod from its pre-tensioned state, to a non-blocking position in which the blocking surface is distant from the position restricting movement of the holding member,
wherein the drive spring is at least partly housed in the tubular plunger rod and is operatively connected to the distal end of the housing.

17. The subassembly according to claim 16, wherein the at least one holding member comprises a proximally extending holding arm arranged to lock the plunger rod in the pre-tensioned state.

18. The subassembly according to claim 17, wherein the proximally extending holding arm is radially displaceable in a direction away from the plunger rod when the plunger holder assumes the non-blocking position.

19. The subassembly according to claim 17, wherein the proximally extending holding arm comprises a surface portion inclined radially towards the tubular plunger rod in the proximal direction.

20. The subassembly according to claim 16, wherein the at least one holding member comprises a circumferentially arranged holding ring having a protrusion extending radially towards the plunger rod, the protrusion being arranged to lock the plunger rod in the pre-tensioned state.

21. The subassembly according to claim 20, wherein the circumferentially arranged holding ring is radially displaceable in a direction away from the plunger rod when the plunger holder assumes the non-blocking position.

22. The subassembly according to claim 16, wherein the tubular plunger rod comprises an engagement member configured to engage with the holding member when the plunger holder assumes the blocking position.

23. The subassembly according to claim 22, wherein the engagement member comprises a protrusion arranged on an envelope surface of the tubular plunger rod, the protrusion extending in a radial and an axial direction.

24. The subassembly according to claim 23, wherein the protrusion comprises a surface portion inclined radially and distally.

25. The subassembly according to claim 22, wherein the engagement member comprises an indentation in an envelope surface of the tubular plunger rod, the holding member being configured to engage with the indentation when the plunger holder assumes the blocking position.

26. The subassembly according to claim 16, wherein the subassembly further comprises a tubular ring member housed by, and fixedly attached to, the actuator housing, the tubular ring member comprising a resilient tongue having an abutment surface arranged in abutment with an envelope surface of the tubular plunger rod.

27. The subassembly according to claim 26, wherein the resilient tongue extends radially and circumferentially towards the tubular plunger rod.

28. The subassembly according to claim 16, wherein the subassembly further comprises a delivery member cover arranged in the housing and extending proximally from the proximal end of the housing, the delivery member cover being configured to, during the medicament delivery operation, move linearly relative to the housing from a protracted position to a retracted position in which the delivery member cover is received further in the housing.

29. The subassembly according to claim 16, wherein the subassembly further comprises a safety clip releasably attached to the housing, the safety clip comprises a first radially extending hook, wherein the safety clip is operatively arranged to, upon activation, move from a first state in which the first radially extending hook is abutting a distal end of the plunger holder, to a second state in which the first radially extending hook is displaced radially away from the plunger holder to thereby allow the plunger holder to transition from the blocking position to the non-blocking position.

30. A medicament delivery device for expelling medicament from a medicament container via a delivery member, the medicament delivery device comprising the subassembly according to claim 16.
